(19) United States
(12) Patent Application Publication
(10) Pub. No.: US 2023/0355387 A1
(43) Pub. Date: Nov. 9, 2023

(54) MECHANICALLY EXPANDABLE BI-CAVAL DOCKING DEVICE

(71) Applicant: EDWARDS LIFESCIENCES CORPORATION, Irvine, CA (US)

(72) Inventor: Anatoly Dvorsky, Haifa (IL)

(21) Appl. No.: 18/355,367

(22) Filed: Jul. 19, 2023

Related U.S. Application Data
(63) Continuation of application No. PCT/US2022/012881, filed on Jan. 19, 2022.
(60) Provisional application No. 63/139,575, filed on Jan. 20, 2021.

(51) Int. Cl. A61F 2/24

(52) U.S. Cl. CPC A61F 2/243; A61F 2250/0048

(57) ABSTRACT

A docking device can include a radially expandable and compressible frame and an actuator. The frame can include a first sub-frame having a first plurality of struts pivotably coupled to one another, a second sub-frame having a second plurality of struts pivotably coupled to one another, and one or more linking struts extending from a first end of the frame to a second end of the frame and coupling the first sub-frame and the second sub-frame to one another. The actuator can include a first member coupled to the frame at a first location and a second member coupled to the frame at a second location axially spaced from the first location. The actuator can be used to mechanically expand the docking device.

### CROSS-REFERENCE TO RELATED APPLICATION

This is a continuation of PCT application no. PCT/US2022/012881 filed on Jan. 19, 2022, which claims the benefit of U.S. Provisional Application No. 63/139,575 filed Jan. 20, 2021, each of which is incorporated in its entirety herein by this specific reference.

### FIELD

The present disclosure relates to implantable, mechanically expandable docking devices and to methods and delivery assemblies for, and including, such docking devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Such transcatheter heart valves (THVs) typically can be implanted directly within a native aortic valve without additional docking devices or anchoring frames. However, in relatively larger native valves or deployment positions, such as the mitral valve, tricuspid valve, the pulmonary artery, the inferior vena cava or superior vena cava, a THV designed for the aortic position typically is too small to secure within the relatively implantation site. In such cases, the THV can be implanted within a larger docking device that is implanted prior to the THV. In other cases, the THV can be provided with an inner frame for supporting prosthetic leaflets and a larger outer anchoring frame for anchoring against the surrounding tissue.

Prosthetic heart valves that rely on a mechanical actuator for expansion can be referred to as “mechanically expandable” devices. Mechanically expandable devices can provide one or more advantages over self-expandable and balloon-expandable prosthetic heart valves. For example, mechanically expandable devices can be expanded to various diameters. Mechanically expandable prosthetic devices can also be compressed after an initial expansion (e.g., for repositioning and/or retrieval).

Despite recent advancements in percutaneous valve technology, there remains a need for improved THVs and/or docking devices allowing THVs to be implanted within larger implantation sites.

### SUMMARY

Described herein are examples of prosthetic heart valves, docking devices, frames, or stations for prosthetic heart valves, docking device sub-frames, and methods for assembling and/or implanting prosthetic heart valves and docking devices. The docking devices can include one or more expansion and locking mechanisms, allowing the docking devices to be mechanically expanded at the implantation site. As a result, the docking devices may be expanded such that any sub-frames that make up a respective docking device expand simultaneously without requiring substantial modification of the delivery apparatus.

In a representative example, a docking device can comprise a radially expandable and compressible frame comprising a first sub-frame comprising a first plurality of struts pivotably coupled to one another, a second sub-frame comprising a second plurality of struts pivotably coupled to one another, and one or more linking struts. The one or more linking struts can extend from a first end of the frame to a second end of the frame and coupling the first sub-frame and the second sub-frame to one another. The docking device can further comprise an actuator comprising a first member coupled to the frame at a first location and a second member coupled to the frame at a second location axially spaced from the first location.

In another representative example, a docking device can comprise a radially expandable and compressible frame including first and second sub-frames spaced axially apart from one another. Each sub-frame can comprise a plurality of struts pivotably coupled to one another at a plurality of junctions defining a single row of cells extending circumferentially around the frame. The plurality of struts can comprise one or more linking struts extending from a first end of the frame to a second end of the frame and coupling the first and second sub-frames to one another. The docking device can further comprise one or more expansion and locking mechanisms each comprising an outer member coupled to the second sub-frame at a first junction, an inner member coupled to the second sub-frame at a second junction axially spaced from the first junction, and a locking member configured to retain the second sub-frame in an expanded configuration.

In still another representative example, a docking device can comprise a radially expandable and compressible frame including a controlling sub-frame and a controlled sub-frame coupled to the controlling sub-frame via one or more linking struts extending from an inflow end of the frame to an outflow end of the frame. The device can further comprise an actuator comprising a first member coupled to the controlling sub-frame at a first location and a second member coupled to the controlling sub-frame at a second location axially spaced from the first location. Movement of the first member and the second member relative to one another in a first direction can apply an expansion force to the controlling sub-frame to cause radial expansion of the controlling sub-frame. The linking struts are configured to transfer the expansion force to the controlled sub-frame such that the controlling sub-frame and the controlled sub-frame expand simultaneously.

In another representative example, a docking device can comprise a radially expandable and compressible frame comprising a first sub-frame comprising a first plurality of struts pivotably coupled to one another, a second sub-frame comprising a second plurality of struts pivotably coupled to one another, and one or more linking struts. The linking struts can extend from an inflow end of the frame to an outflow end of the frame and coupling the first sub-frame and the second sub-frame to one another. The device can further comprise first and second expansion and locking mechanisms each comprising an outer member and an inner member extending at least partially into the outer member. The first expansion and locking mechanism can be coupled to the first sub-frame and the second expansion and locking mechanism can be coupled to the second sub-frame such that the first and second sub-frames are radially expandable and compressible independently from one another.

In a representative example, an assembly can comprise a docking device and a delivery apparatus. The docking device can comprise a radially expandable and compressible frame including first and second sub-frames spaced axially apart from one another, each sub-frame comprising a plurality of struts pivotably coupled to one another at a plurality of junctions defining a single row of cells extending circumferentially around the frame. The plurality of struts can comprise one or more linking struts extending from an inflow end of the first sub-frame to an inflow end of the second sub-frame and coupling the first and second sub-frames to one another. The docking device can further comprise one or more expansion and locking mechanisms each comprising an outer member coupled to the second sub-frame at a first junction, an inner member coupled to the second sub-frame at a second junction axially spaced from the first junction, and a locking member configured to retain the second sub-frame in an expanded configuration. The delivery apparatus can comprise a handle, a first actuation member extending from the handle and coupled to the outer member, the first actuation member configured to apply a first expansion force to the first member, and a second actuation member extending from the handle and coupled to the inner member, the second actuation member configured to apply a second expansion force to the second member. Application of the first and second expansion forces via the first and second actuation members causes radial expansion of the second sub-frame and the first sub-frame simultaneously.

In another representative example, an assembly can comprise a docking device and a delivery apparatus. The docking device can comprise a radially expandable and compressible frame including first and second sub-frames spaced axially apart from one another. Each sub-frame can comprise a plurality of struts pivotably coupled to one another at a plurality of junctions defining a single row of cells extending circumferentially around the frame. The plurality of struts can include one or more linking struts extending from an inflow end of the first sub-frame to an inflow end of the second sub-frame and coupling the first and second sub-frames to one another. The device can further include first and second expansion and locking mechanisms each comprising an outer member and an inner member extending at least partially into the outer member. The first expansion and locking mechanism can be coupled to the first sub-frame and the second expansion and locking mechanism can be coupled to the second sub-frame. The delivery apparatus can comprise a handle, first and second actuation assemblies extending from the handle coupled to respective expansion and locking mechanisms. Each actuation assembly can be configured to apply an expansion force to a respective expansion and locking mechanism. Application of a first expansion force to the first sub-frame via the first actuation assembly can causes radial expansion of the first sub-frame independently of the second sub-frame, and application of a second expansion force to the second sub-frame via the second actuation assembly can cause radial expansion of the second sub-frame independently of the first sub-frame.

In still another representative example, an assembly can comprise a docking device, a delivery apparatus, and a prosthetic valve. The docking device can comprise a radially expandable and compressible frame including first and second sub-frames spaced axially apart from one another. Each sub-frame can comprise a plurality of struts pivotably coupled to one another at a plurality of junctions defining a single row of cells extending circumferentially around the frame. The plurality of struts can comprise one or more linking struts extending from an inflow end of the first sub-frame to an inflow end of the second sub-frame and coupling the first and second sub-frames to one another. The device can further comprise one or more expansion and locking mechanisms each comprising an outer member coupled to the second sub-frame at a first junction, an inner member coupled to the second sub-frame at a second junction axially spaced from the first junction, and a locking member configured to retain the second sub-frame in an expanded configuration. The delivery apparatus can comprise a handle, a first actuation member extending from the handle and coupled to the outer member, the first actuation member configured to apply a first expansion force to the first member, and a second actuation member extending from the handle and coupled to the inner member, the second actuation member configured to apply a second expansion force to the second member, wherein application of the first and second expansion forces via the first and second actuation members causes radial expansion of the second sub-frame and the first sub-frame simultaneously. The prosthetic valve can be disposed within the second sub-frame and can comprise a radially expandable and compressible frame and a valvular structure disposed within and coupled to the frame.

In a representative example, a method can comprise inserting a distal end portion of a delivery apparatus into a vasculature of a patient. The delivery apparatus can be releasably coupled to a docking device movable between a radially compressed and a radially expanded configuration. The docking device can comprise a frame including first and second sub-frames spaced axially apart from one another and one or more actuators. Each sub-frame can comprise a plurality of struts pivotably coupled to one another at a plurality of junctions and including one or more linking struts extending from an inflow end of the first sub-frame to an inflow end of the second sub-frame and coupling the first and second sub-frames to one another. The method can further comprise advancing the docking device to a selected implantation site, and actuating the actuators to radially expand the second sub-frame such that the expansion force is transferred to the first sub-frame via the linking struts allowing the first and second sub-frames to expand simultaneously.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a perspective view of a prosthetic heart valve, according to one example.

FIG. 2 is a side elevation view of a delivery apparatus for a prosthetic heart valve, according to one example.

FIG. 3 is a perspective view of a docking device shown in an expanded configuration, according to one example.

FIG. 4 is a side elevation view of the docking device of FIG. 3 shown in a compressed configuration.

FIG. 5 is a perspective view of the docking device of FIG. 3 shown in an expanded configuration.

FIG. 6 is a perspective view of the docking device of FIG. 3 shown in an expanded configuration and including an expansion and locking mechanism, according to one example.

FIG. 7 is a cutaway view of the human heart including the docking device of FIG. 3 extending from the superior vena cava to the inferior vena cava.

FIG. 8 is a perspective view of a docking device shown in an expanded configuration, according to one example.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

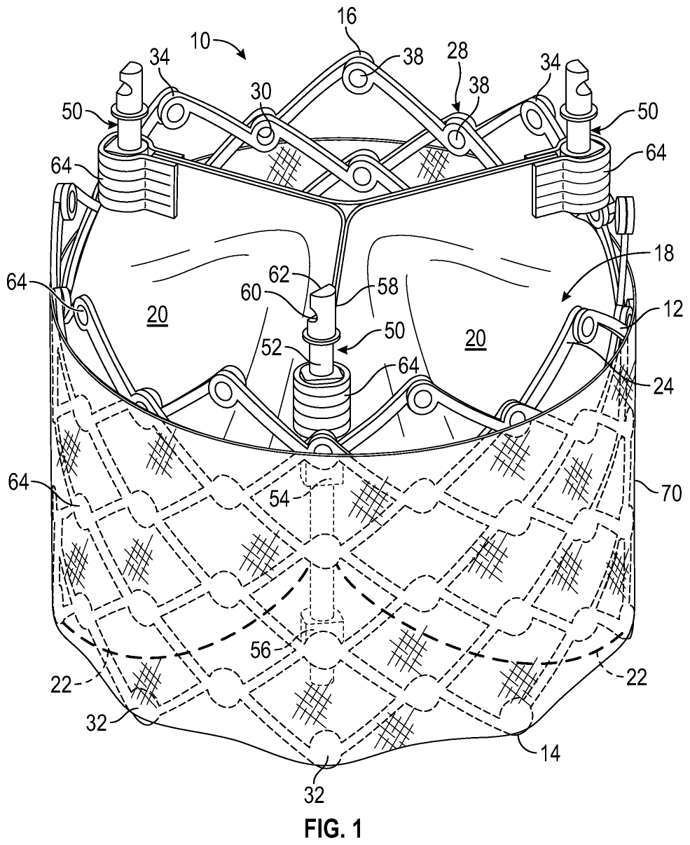

All features described herein are independent of one another and, except where structurally impossible, can be used in combination with any other feature described herein. For example, an actuator 50 as shown in FIG. 1 can be used in combination with docking devices 200 or 400. In another example, a valvular structure 18 as shown in FIG. 1 can be used in combination with the docking devices 200 or 400 and can be coupled to the first and/or second sub-frames.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

Examples of the Disclosed Technology

Described herein are examples of the present disclosure directed to devices and methods for providing a docking device/docking station for a prosthetic valve (e.g., a transcatheter heart valve) such as prosthetic valve 10. Though the docking devices described herein are described and/or illustrated as being used within the superior vena cava (SVC) and/or the inferior vena cava (IVC), it should be appreciated that the docking devices and/or prosthetic valves described herein can also be used in other areas of the anatomy, heart, or vasculature, such as the tricuspid valve, the pulmonary valve, the pulmonary artery, the aortic valve, the aorta, the mitral valve, or other locations. The docking devices described herein can be configured to compensate for the deployed prosthetic valve having a smaller diameter and/or different geometric shape than the implantation site. For example, the native anatomy of the IVC can be ovular or egg-shaped while the prosthetic valve can be cylindrical.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the valves disclosed herein may be used with a variety of implant delivery apparatuses, and examples thereof will be discussed in more detail later.

FIG. 1 shows an exemplary prosthetic valve 10, according to one example. The prosthetic valve 10 can include an annular stent or frame 12 having an inflow end 14 and an outflow end 16. The prosthetic valve 10 can also include a valvular structure 18 which is coupled to and supported inside of the frame 12. The valvular structure 18 is configured to regulate the flow of blood through the prosthetic valve 10 from the inflow end 14 to the outflow end 16.

The valvular structure 18 can include, for example, a leaflet assembly comprising one or more leaflets 20 made of a flexible material. The leaflets 20 can be made from in whole or part, biological material, bio-compatible synthetic materials, or other such materials. Suitable biological material can include, for example, bovine pericardium (or pericardium from other sources). The leaflets 20 can be secured to one another at their adjacent sides to form commissures, each of which can be secured to a respective actuator 50 or the frame 12.

In the depicted example, the valvular structure 18 comprises three leaflets 20, which can be arranged to collapse in a tricuspid arrangement. Each leaflet 20 can have an inflow edge portion 22. As shown in FIG. 1, the inflow edge portions 22 of the leaflets 20 can define an undulating, curved scallop shape that follows or tracks a plurality of interconnected strut segments of the frame 12 in a circumferential direction when the frame 12 is in the radially expanded configuration. The inflow edges of the leaflets can be referred to as a “scallop line.”

In some examples, the inflow edge portions 22 of the leaflets 20 can be sutured to adjacent struts of the frame generally along the scallop line. In other examples, the inflow edge portions 22 of the leaflets 20 can be sutured to an inner skirt, which in turn in sutured to adjacent struts of the frame. By forming the leaflets 20 with this scallop geometry, stresses on the leaflets 20 are reduced, which in turn improves durability of the valve 10. Moreover, by virtue of the scallop shape, folds and ripples at the belly of each leaflet 20 (the central region of each leaflet), which can cause early calcification in those areas, can be eliminated or at least minimized. The scallop geometry also reduces the amount of tissue material used to form valvular structure 18, thereby allowing a smaller, more even crimped profile at the inflow end 14 of the valve 10.

Further details regarding transcatheter prosthetic heart valves, including the manner in which the valvular structure can be mounted to the frame of the prosthetic valve can be found, for example, in U.S. Pat. Nos. 6,730,118, 7,393,360, 7,510,575, 7,993,394, 8,252,202, and 11,135,056 and U.S. Publication No. 2020/0352711, all of which are incorporated herein by reference in their entireties.

The prosthetic valve 10 can be radially compressible and expandable between a radially compressed configuration and a radially expanded configuration. The frame 12 can include a plurality of interconnected lattice struts 24 arranged in a lattice-type pattern and forming a plurality of apices 34 at the outflow end 16 of the prosthetic valve 10. The struts 24 can also form similar apices 32 at the inflow end 14 of the prosthetic valve 10.

The struts 24 can be pivotably coupled to one another at one or more pivot joints or pivot junctions 28 along the length of each strut. For example, in one example, each of the struts 24 can be formed with apertures 30 at opposing ends of the strut and apertures spaced along the length of the strut. Respective hinges can be formed at the locations where struts 24 overlap each other via fasteners 38, such as rivets or pins that extend through the apertures 30. The hinges can allow the struts 24 to pivot relative to one another as the frame 12 is radially expanded or compressed, such as during assembly, preparation, or implantation of the prosthetic valve 10.

The frame struts and the components used to form the pivot joints of the frame 12 (or any frames described below) can be made of any of various suitable materials, such as stainless steel, a cobalt chromium alloy, or a nickel titanium alloy (“NiTi”), for example Nitinol. In some examples, the frame 12 can be constructed by forming individual components (e.g., the struts and fasteners of the frame) and then mechanically assembling and connecting the individual components together. Further details regarding the construction of the frame and the prosthetic valve are described in U.S. Pat. Nos. 10,603,165, 10,869,759, and 10,806,573 and U.S. Patent Publication No. 2020/00188099, all of which are incorporated herein by reference.

In the illustrated example, the prosthetic valve 10 can be mechanically expanded from the radially contracted configuration to the radially expanded configuration. For example, the prosthetic valve 10 can be radially expanded by maintaining the inflow end 14 of the frame 12 at a fixed position while applying a force in the axial direction against the outflow end 16 toward the inflow end 14. Alternatively, the prosthetic valve 10 can be expanded by applying an axial force against the inflow end 14 while maintaining the outflow end 16 at a fixed position, or by applying opposing axial forces to the inflow and outflow ends 14, 16, respectively.

As shown in FIG. 1, the prosthetic valve 10 can include one or more actuators 50 mounted to and equally spaced around the inner surface of the frame 12. Each of the actuators 50 can be configured to form a releasable connection with one or more respective actuators of a delivery apparatus.

In the illustrated example, expansion and compression forces can be applied to the frame by the actuators 50. Referring again to FIG. 1, each of the actuators 50 can comprise a screw or threaded rod 52, a first anchor in the form of a cylinder or sleeve 54, and a second anchor in the form of a threaded nut 56. The rod 52 extends through the sleeve 54 and the nut 56. The sleeve 54 can be secured to the frame 12, such as with a fastener 38 that forms a hinge at the junction between two struts. Each actuator 50 is configured to increase the distance between the attachment locations of a respective sleeve 54 and nut 56, which causes the frame 12 to elongate axially and compress radially, and to decrease the distance between the attachment locations of a respective sleeve 54 and nut 56, which causes the frame 12 to foreshorten axially and expand radially.

For example, each rod 52 can have external threads that engage internal threads of the nut 56 such that rotation of the rod causes corresponding axial movement of the nut 56 toward or away from the sleeve 54 (depending on the direction of rotation of the rod 52). This causes the hinges supporting the sleeve 54 and the nut 56 to move closer towards each other to radially expand the frame or to move farther away from each other to radially compress the frame, depending on the direction of rotation of the rod 52.

In other examples, the actuators 50 can be reciprocating type actuators configured to apply axial directed forces to the frame to produce radial expansion and compression of the frame. For example, the rod 52 of each actuator can be fixed axially relative to the nut 56 and slidable relative to the sleeve 54. Thus, in this manner, moving the rod 52 distally relative to the sleeve 54 and/or moving the sleeve 54 proximally relative to the rod 52 radially compresses the frame. Conversely, moving the rod 52 proximally relative to the sleeve 54 and/or moving the sleeve 54 distally relative to the rod 52 radially expands the frame.

When reciprocating type actuators are used, the prosthetic valve can also include one or more locking mechanisms that retain the frame in the expanded state. The locking mechanisms can be separate components that are mounted on the frame apart from the actuators, or they can be a sub-component of the actuators themselves.

Each rod 52 can include an attachment member 58 along a proximal end portion of the rod 52 configured to form a releasable connection with a corresponding actuator of a delivery apparatus. The actuator(s) of the delivery apparatus can apply forces to the rods for radially compressing or expanding the prosthetic valve 10. The attachment member 58 in the illustrated configuration comprises a notch 60 and a projection 62 that can engage a corresponding projection of an actuator of the delivery apparatus.

In the illustrated examples, the prosthetic valve 10 includes three such actuators 50, although a greater or fewer number of actuators could be used in other examples. The leaflets 20 can have commissure attachments members 64 that wrap around the sleeves 54 of the actuators 50. Further details of the actuators, locking mechanisms and delivery apparatuses for actuating the actuators can be found in U.S. Pat. Nos. 10,603,165, 10,806,573, and 11,135,056, and International Application Nos. PCT/US2020/057691 and PCT/US2021/022467, each of which is incorporated herein by reference in its entirety. Any of the actuators and locking mechanisms disclosed in the previously filed applications can be incorporated in any of the prosthetic valves disclosed herein. Further, any of the delivery apparatuses disclosed in the previously filed applications can be used to deliver and implant any of the prosthetic valves discloses herein.

The prosthetic valve 10 can include one or more skirts or sealing members. In some examples, the prosthetic valve 10 can include an inner skirt (not shown) mounted on the inner surface of the frame. The inner skirt can function as a sealing member to prevent or decrease perivalvular leakage, to anchor the leaflets to the frame, and/or to protect the leaflets against damage caused by contact with the frame during crimping and during working cycles of the prosthetic valve. As shown in FIG. 1, the prosthetic valve 10 can also include an outer skirt 70 mounted on the outer surface of the frame 12. The outer skirt 70 can function as a sealing member for the prosthetic valve by sealing against the tissue of the native valve annulus and helping to reduce paravalvular leakage past the prosthetic valve. The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valve can be found, for example, in U.S. Patent Publication No. 2020/0352711, which is incorporated herein by reference in its entirety.

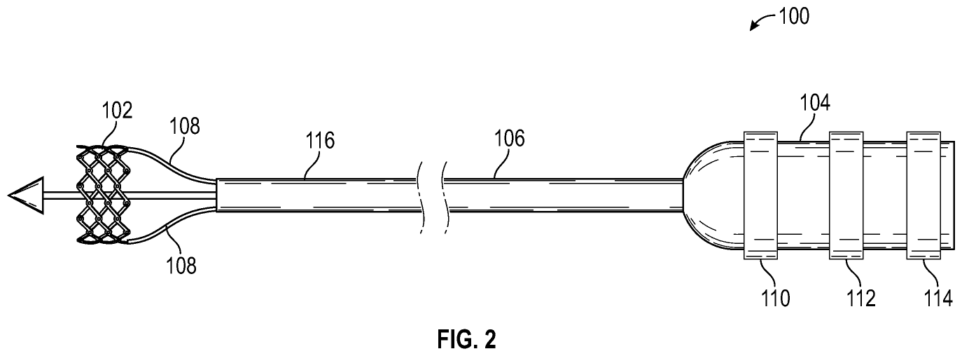

FIG. 2 illustrates a delivery apparatus 100, according to one example, adapted to deliver a prosthetic heart valve 102, such as the illustrated prosthetic heart valve 10, described above. The prosthetic valve 102 can be releasably coupled to the delivery apparatus 100. It should be understood that the delivery apparatus 100 and other delivery apparatuses disclosed herein can be used to implant prosthetic devices other than prosthetic valves, such as stents or grafts.

The delivery apparatus 100 in the illustrated example generally includes a handle 104, a first elongated shaft 106 (which comprises an outer shaft in the illustrated example) extending distally from the handle 104, at least one actuator assembly 108 extending distally through the outer shaft 106. The at least one actuator assembly 108 can be configured to radially expand and/or radially collapse the prosthetic valve 102 when actuated.

Though the illustrated example shows two actuator assemblies 108 for purposes of illustration, it should be understood that one actuator 108 can be provided for each actuator on the prosthetic valve. For example, three actuator assemblies 108 can be provided for a prosthetic valve having three actuators. In other examples, a greater or fewer number of actuator assemblies can be present.

In some examples, a distal end portion 116 of the shaft 106 can be sized to house the prosthetic valve in its radially compressed, delivery state during delivery of the prosthetic valve through the patient's vasculature. In this manner, the distal end portion 116 functions as a delivery sheath or capsule for the prosthetic valve during delivery,

The actuator assemblies 108 can be releasably coupled to the prosthetic valve 102. For example, in the illustrated example, each actuator assembly 108 can be coupled to a respective actuator of the prosthetic valve 102. Each actuator assembly 108 can comprise a support tube, an actuator member, and a locking tool. When actuated, the actuator assembly can transmit pushing and/or pulling forces to portions of the prosthetic valve to radially expand and collapse the prosthetic valve as previously described. The actuator assemblies 108 can be at least partially disposed radially within, and extend axially through, one or more lumens of the outer shaft 106. For example, the actuator assemblies 108 can extend through a central lumen of the shaft 106 or through separate respective lumens formed in the shaft 106.

The handle 104 of the delivery apparatus 100 can include one or more control mechanisms (e.g., knobs or other actuating mechanisms) for controlling different components of the delivery apparatus 100 in order to expand and/or deploy the prosthetic valve 102. For example, in the illustrated example the handle 104 comprises first, second, and third knobs 110, 112, and 114.

The first knob 110 can be a rotatable knob configured to produce axial movement of the outer shaft 106 relative to the prosthetic valve 102 in the distal and/or proximal directions in order to deploy the prosthetic valve from the delivery sheath 116 once the prosthetic valve has been advanced to a location at or adjacent the desired implantation location with the patient's body. For example, rotation of the first knob 110 in a first direction (e.g., clockwise) can retract the sheath 116 proximally relative to the prosthetic valve 102 and rotation of the first knob 110 in a second direction (e.g., counter-clockwise) can advance the sheath 116 distally. In other examples, the first knob 110 can be actuated by sliding or moving the knob 110 axially, such as pulling and/or pushing the knob. In other examples, actuation of the first knob 110 (rotation or sliding movement of the knob 110) can produce axial movement of the actuator assemblies 108 (and therefore the prosthetic valve 102) relative to the delivery sheath 116 to advance the prosthetic valve distally from the sheath 116.

The second knob 112 can be a rotatable knob configured to produce radial expansion and/or contraction of the prosthetic valve 102. For example, rotation of the second knob 112 can move the actuator member and the support tube axially relative to one another. Rotation of the second knob 112 in a first direction (e.g., clockwise) can radially expand the prosthetic valve 102 and rotation of the second knob 112 in a second direction (e.g., counter-clockwise) can radially collapse the prosthetic valve 102. In other examples, the second knob 112 can be actuated by sliding or moving the knob 112 axially, such as pulling and/or pushing the knob.

The third knob 114 can be a rotatable knob configured to retain the prosthetic heart valve 102 in its expanded configuration. For example, the third knob 114 can be operatively connected to a proximal end portion of the locking tool of each actuator assembly 108. Rotation of the third knob in a first direction (e.g., clockwise) can rotate each locking tool to advance the locking nuts to their distal positions to resist radial compression of the frame of the prosthetic valve, as described above. Rotation of the knob 114 in the opposite direction (e.g., counterclockwise) can rotate each locking tool in the opposite direction to decouple each locking tool from the prosthetic valve 102. In other examples, the third knob 114 can be actuated by sliding or moving the third knob 114 axially, such as pulling and/or pushing the knob.

Although not shown, the handle 104 can include a fourth rotatable knob operative connected to a proximal end portion of each actuator member. The fourth knob can be configured to rotate each actuator member, upon rotation of the knob, to unscrew each actuator member from the proximal portion of a respective actuator. As described above, once the locking tools and the actuator members are uncoupled from the prosthetic valve 102, they can be removed from the patient.

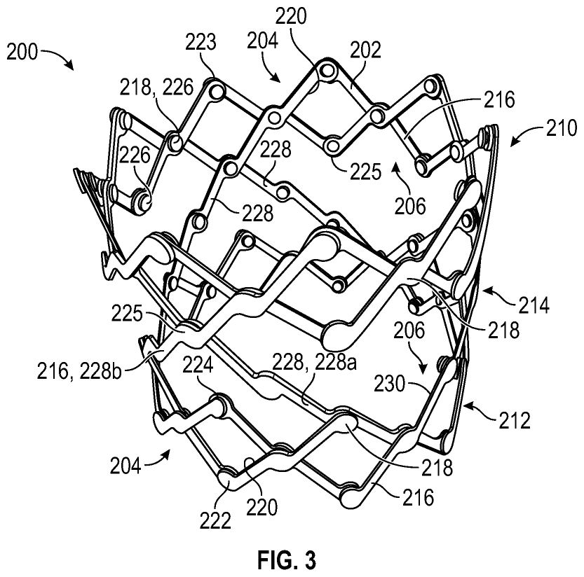

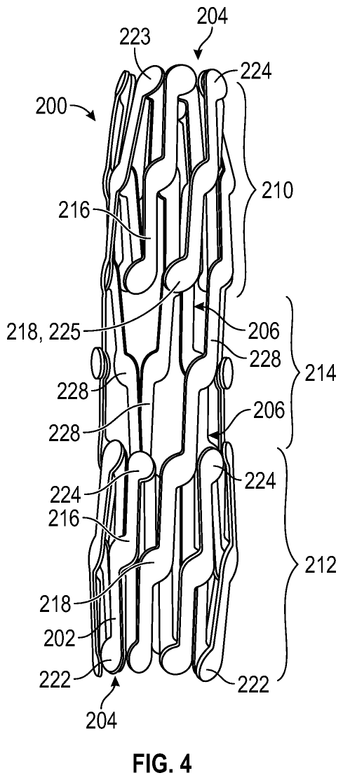

FIGS. 3-6 illustrate an exemplary example of a docking device 200. Docking device 200 can be configured to compensate for a deployed prosthetic valve (e.g., prosthetic valve 10 described previously) having a smaller diameter and/or different geometric shape than the implantation site.

Various examples of docking devices and examples of prosthetic valves are disclosed herein, and any combination of these options can be made unless specifically excluded. That is, any of the docking devices disclosed can be used with any type of valve and/or any delivery system, even if a specific combination is not explicitly described. For example, the docking devices described herein can be used to secure any of various mechanically-expandable valves, such as the prosthetic valves described in U.S. Pat. No. 10,603,165 and International Application No. PCT/US2021/052745, each of which is incorporated herein by reference. For example, some mechanical valves can comprise pivotable junctions between the struts (such as prosthetic valve 10 and the prosthetic valves disclosed in U.S. Pat. No. 10,603,165), while others can comprise a unitary lattice frame expandable and/or compressible via mechanical means (such as the prosthetic valves disclosed in International Application No. PCT/US2021/052745). However, it should be appreciated that the docking devices can additionally be used with other types of transcatheter prosthetic valves, including balloon-expandable prosthetic heart valves, such as disclosed in U.S. Pat. Nos., 9,393,110, and 11,096,781, and U.S. Publication No. 2019/0365530, each of which are incorporated herein by reference, and self-expandable prosthetic heart valves, such as disclosed in U.S. Pat. No. 10,098,734, which is incorporated herein by reference.

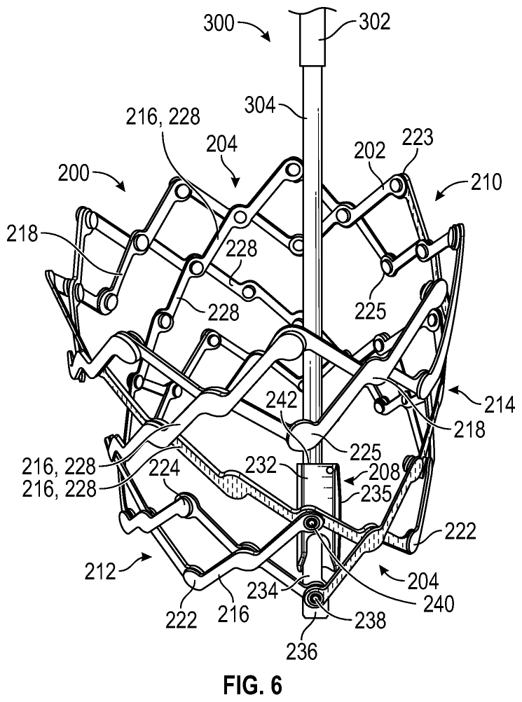

Referring to FIG. 3, docking device 200 can generally comprise a radially expandable and compressible frame 202, and a one or more expansion and locking mechanisms 208 (FIG. 6) configured to radially expand and/or compress the frame 202 and lock the frame in the expanded configuration. The frame 202 can be configured as a bi-caval frame comprising a first sub-frame 210 and a second sub-frame 212 coupled together via a linking or intermediate portion 214. Each sub-frame 210, 212 can include an inflow end portion 204 and an outflow end portion 206.

The first and second sub-frames 210, 212 can each comprise a plurality of struts 216 pivotably connected to each other at a plurality of junctions 218 that permit pivoting of the struts relative to one another when the docking device 200 is radially compressed and/or expanded. The struts 216 can be arranged in a lattice-type pattern, defining a plurality of cells 220 extending circumferentially around the frame 202 in one or more rows. In the illustrated example, the first sub-frame 210 and the second sub-frame 212 each comprise one row of cells 220. However, in other examples, the sub-frames 210, 212 can comprise a greater number of rows. Each sub-frame 210, 212 can comprise a plurality of inflow apices and outflow apices, for example, sub-frame 210 comprises inflow apices 223 and outflow apices 225 and sub-frame 212 can comprises inflow apices 222 and outflow apices 224.

In the illustrated example, the struts 216 can comprise a plurality of apertures through which fasteners 226 can extend to couple the struts to one another at each junction 218. The fasteners 226 can be, for example, rivets or pins. In other examples, the docking device 200 can comprise a mechanically-expandable unitary lattice frame, such as the frames described in International Application No. PCT/US2021/052745.

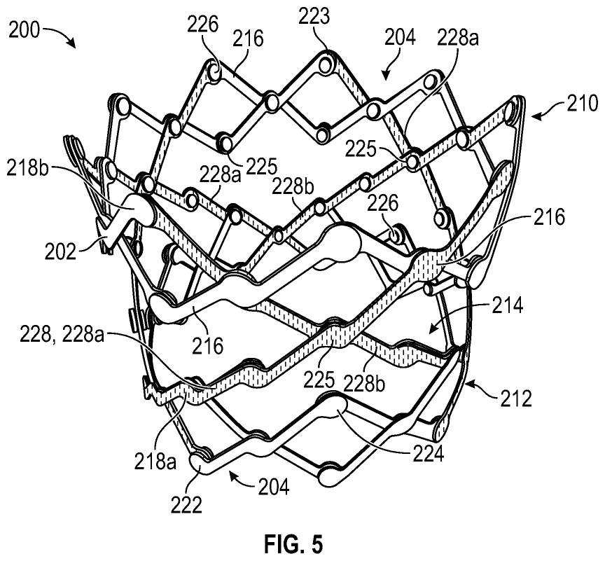

Selected struts 216 of the frame 202 can be configured as linking struts 228 and can extend from the first inflow end portion 204 to the second inflow end portion 204 of the docking device 200, forming the linking portion 214 that couples the first sub-frame 210 and the second sub-frame 212 to one another. The stippling in FIGS. 5-6 is added to distinguish the linking struts 228 from the plurality of struts 216 and does not represent actual surface ornamentation. As shown in FIG. 5, a first sub-set 228a of linking struts 228 can extend in a first direction, and a second sub-set 228b of linking struts can extend in a second direction such that respective struts of the first and second sets 228a, 228b can overlap one another at selected inflow apices 222 of the first sub-frame 210. Referring again to FIG. 3, the linking struts 228 can define a plurality of larger cells 230 (relative to the cells of the first and second sub-frames 210, 212) configured such that, after the docking device has been implanted at a selected implantation site, the flow of blood through the linking portion 214 is unimpacted.

One or both of the sub-frames 210, 212 can be configured as a docking station (e.g., configured to receive and retain a prosthetic valve, such as prosthetic valve 10 described above). In examples where only one sub-frame is configured as a docking station, the remaining sub-frame can be used to stabilize the docking device 200 at the implantation site.

In some examples, the docking device 200 can include one or more outer skirts and/or sealing members. The sealing members can extend circumferentially around the first and/or second sub-frames and can be configured to expand radially outwardly to help secure the one or more sub-frames at the implantation site. In some examples, the sealing members can comprise, for example, fabric, cloth, foam, etc. In some examples, the docking device 200 can include an inner skirt or inner sealing member positioned on the inner surface of the frame 202, such as for promoting a fluid-tight seal between the frame 202 and a prosthetic valve implanted within one or both of the sub-frames 210, 212. Further details of skirts and seals that can be incorporated in the docking device 200 (and other examples of docking devices disclosed herein) are disclosed in U.S. Pat. No. 10,363,130 and U.S. Publication No. 2019/0000615, which are incorporated herein by reference.

Referring now to FIG. 6, the frame 202 be configured as a mechanically-expandable frame and can comprise one or more expansion and locking mechanisms 208 (also referred to as “actuators”) configured to both radially expand and lock the docking device 200 in the radially expanded state. Though FIG. 6 shows only a single expansion and locking mechanism 208 mounted to the docking device 200, it should be appreciated that the docking device 200 can comprise any number of expansion and locking mechanisms 208. For example, in some examples, a docking device 200 can comprise two expansion and locking mechanisms 208, or three expansion and locking mechanisms, or four expansion and locking mechanisms, etc. The expansion and locking mechanisms 208 can be placed at any position about the circumference of the frame 202. For example, in some examples, the expansion and locking mechanisms 208 can be equally spaced from one another about the circumference of the frame 202. In other examples, it can be advantageous to have two or more expansion and locking mechanisms 208 situated adjacent to one another.

Expansion and locking mechanism 208 can comprise a first or outer member 232 having an inner bore and a second or inner member 234 extending at least partially into the outer member 232. A first end portion 236 of the inner member 234 can be coupled to the frame 202 at a first location via a fastener 238 that is affixed to and extends radially from the first end portion 236 of the inner member 234. The fastener 238 can be, for example, a rivet or pin. As shown, in some examples, the fastener 238 can extend through corresponding openings at a junction 218 of two overlapping struts 216 and can serve as a pivot pin around which the struts 216 can pivot relative to each other and the inner member 234. The outer member 232 can be coupled to the frame 202 at a second location, axially spaced from and circumferentially aligned with the first location, such as via a fastener 240 (e.g., a rivet or pin). The fastener 240 is affixed to and extends radially from the outer member 232 through a junction of two overlapping struts 216 and can serve as a pivot pin around which the struts 216 can pivot relative to each other and the outer member 232.

As shown in FIG. 6, in some examples, the expansion and locking mechanism 208 can further comprise a locking member 235 configured to lock the outer member 232 and inner member 234 such that they are restrained from movement relative to one another in one or more directions, thereby locking the frame 202 at a selected diameter. For example, using a ratchet mechanism such as a rack and pawl mechanism. In other examples, the inner member 234 can comprise a threaded screw that engages a correspondingly threaded portion within the outer member 232. Further details of expansion and locking mechanisms can be found, for example, in U.S. Pat. No. 10,603,165 and International Publication No. WO2021/086933, each of which is incorporated herein by reference in its entirety. Any of the described expansion and locking mechanisms described therein be used with docking device 200.

Referring still to FIG. 6, as shown in the illustrated example, the inner member 234 is secured to the second sub-frame 212 near the distal or inflow end 204 (e.g., at an inflow apex 222) and the outer member 234 is secured to the second sub-frame 212 near the proximal or outflow end of the second sub-frame 212 (e.g., at an outflow apex 224 of the second sub-frame). The inner member 234 can be axially movable relative to the outer member 232 in a proximal and/or distal direction. As such, because the inner member 234 and the outer member 232 are secured to the second sub-frame 212 at axially spaced locations, moving the inner member 234 and the outer member 232 axially with respect to one another in a telescoping manner can cause radial expansion or compression of the second sub-frame 212. For example, moving the inner member 234 toward the outflow end 206 of the second sub-frame 212 while holding the outer member 232 in a fixed position and/or moving the outer member 232 distally toward the inflow end 204 of the second sub-frame 212 can cause the second sub-frame 212 to foreshorten axially and expand radially. Conversely, moving the inner member 234 toward the inflow end 204 of the second sub-frame 212 and/or moving the outer member 232 toward the outflow end 206 causes the second sub-frame 212 to elongate axially and compress radially.

As shown in FIG. 6, each of the inner and outer member 232, 234 is coupled to a junction 218 that comprises at least one linking strut 228, shown with a stippled pattern. Such a configuration allows the expansion and/or compression forces applied to the second sub-frame 212 to be transferred to the first sub-frame 210 via the linking struts 228, thereby allowing the first and second sub-frames 210, 212 to be expanded and/or compressed simultaneously. In such examples, the second sub-frame 212 can be referred to as the ‘controlling sub-frame’ and the first sub-frame 210 can be referred to as the ‘controlled sub-frame.’ Accordingly, the expansion and locking mechanism 208 can be used to radially expand the docking device 200 and to lock the docking device 200 in the expanded configuration. In other examples, both the outer and inner members 232, 234 can be coupled to the first sub-frame 210 at axially spaced locations such that the first sub-frame 210 can be the controlling sub-frame and the second sub-frame 212 can be the controlled sub-frame.

In some particular examples, a single expansion and locking mechanism 208 can be used to expand the docking device 200. In such examples, the inner member 234 of the expansion and locking mechanism 208 can be coupled to a junction 218 on the second sub-frame 212 that comprises a linking strut 228 (such as junction 218a shown in FIG. 5), and the outer member 232 can be coupled to a junction 218 on the first sub-frame 210 that comprises a linking strut 228 (such as junction 218b shown in FIG. 5).

Referring still to FIG. 6, the docking device 200 can be coupled to a delivery apparatus (such as delivery apparatus 100 described previously) via the one or more expansion and locking mechanisms 208 in the following exemplary manner. The delivery apparatus can comprise one or more actuation assemblies 300 (e.g., similar to actuation assemblies 108) comprising a first or outer actuation member 302 (also referred to as a support member) and a second or inner actuation member 304. The second actuation member 304 can extend co-axially through the first actuation member 302, as shown. The second actuation member 304 can releasably couple the inner member 234 and the first actuation member 302 can abut an outflow end 242 of the outer member 232. Further details of methods for coupling expansion and locking mechanisms to actuator assemblies can be found, for example, in U.S. Pat. No. 10,603,165, International Publication Nos. WO2021/086933, and WO2021/146101, each of which is incorporated herein by reference in its entirety.

The docking device 200 can be implanted at a selected implantation site in the following exemplary manner. Generally, the docking device 200 is placed in a radially compressed state and releasably coupled to one or more actuator assemblies 300 of a delivery apparatus (such as delivery apparatus 100 shown in FIG. 2), as described above, and the delivery apparatus and docking device can be advanced through the vasculature of a patient to a selected implantation site (e.g., within the IVC and SVC). The docking device 200 can then be deployed at the implantation site, and can be expanded and locked in the expanded configuration using the expansion and locking mechanisms 208.

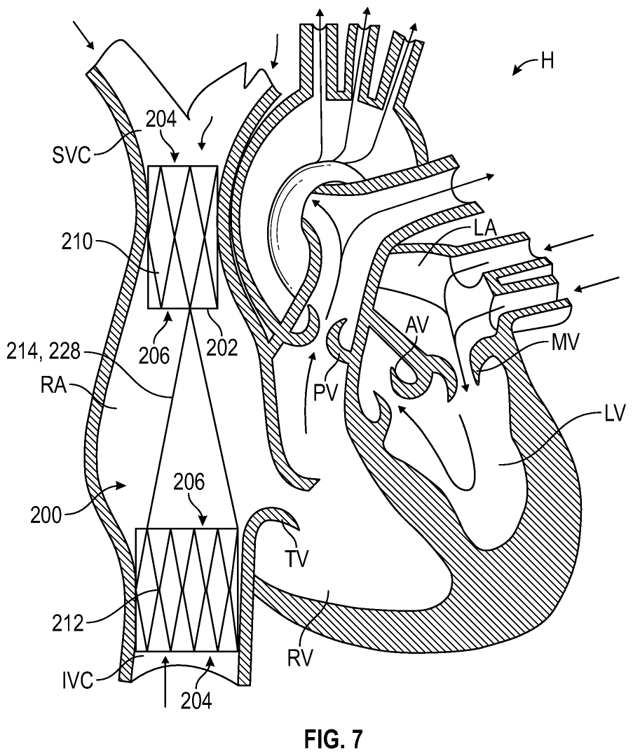

FIG. 7 illustrates a cutaway view of an exemplary human heart H. The right ventricle RV and left ventricle LV are separated from the right atrium RA and left atrium LA, respectively, by the tricuspid valve TV and mitral valve MV, e.g., the atrioventricular valves. The aortic valve AV separates the left ventricle LV from the ascending aorta and the pulmonary valve PV separates the right ventricle RV from the pulmonary artery. As shown in FIG. 7, in the illustrated example, the first sub-frame 210 (e.g., the upper sub-frame in the orientation shown in FIG. 3) can be disposed in the superior vena cava SVC and the second sub-frame 212 (e.g., the lower sub-frame in the orientation shown in FIG. 3) can be disposed in the inferior vena cava IVC. The linking portion 214 can be disposed within the right atrium RA such that the larger cells 230 can cause minimal impact on the blood flow through the right atrium.

To expand the docking device 200, the delivery apparatus can be used to apply a distally directed force to the outer member 232 via the first actuation member 302 and/or a proximally directed force to the inner member 234 via the second actuation member 304 to move the outer member 232 and inner member 234 axially relative to one another in a telescoping manner to cause the second sub-frame 212 to expand. As the second sub-frame 212 expands, the expansion forces are transferred to the first sub-frame 210 via the linking struts 228 such that the first and second sub-frames 210, 212 expand simultaneously. Once a selected diameter for the first and second sub-frames 210, 212 is reached, the delivery apparatus can be uncoupled from the expansion and locking mechanisms 208 and removed from the patient's body.

In some examples, once the docking device 200 has been implanted at the selected implantation site, a prosthetic valve (such as prosthetic valve 10 described previously) can be implanted within the first and/or second sub-frame 210, 212. For example, in one specific implementation, a prosthetic valve is implanted only within the first sub-frame 210 to regulate the flow of blood from the SVC into the right atrium. In another implementation, a prosthetic valve is implanted only within the second sub-frame 12 to regulate the flow of blood from the IVC to the right atrium. In other implementation, prosthetic valves are implanted within the first sub-frame 210 and the second sub-frame 212.

In any case, the prosthetic valve can be placed in a radially compressed state and releasably coupled to the delivery apparatus and the delivery apparatus and prosthetic valve can be advanced through patient's vasculature to the first or second sub-frame 210, 212. So positioned, the prosthetic valve can be radially expanded within the selected sub-frame to dock the prosthetic valve at the selected implantation site.

In other examples, the prosthetic valve can be integrated with the docking device 200 such that it is already disposed within the first and/or second sub-frame 210, 212 prior to and during implantation of the docking device. For example, the docking device 200 can be part of an implantable valve assembly that includes a prosthetic valve (including a respective frame and prosthetic leaflets) pre-assembled or pre-installed within the first and/or second sub-frame 210, 212. A prosthetic valve can be pre-assembled within a sub-frame 210, 212 using sutures, fabrics, welding, or other connection means. In such examples, the prosthetic valve(s) (e.g., prosthetic valve(s) 10) can be expanded during expansion of the docking device 200. In other examples, the first and/or second sub-frames 210, 212 can optionally be formed as a prosthetic valve including a valvular structure (e.g., prosthetic leaflets 20) disposed within and supported directly by the sub-frame without a separate inner frame.

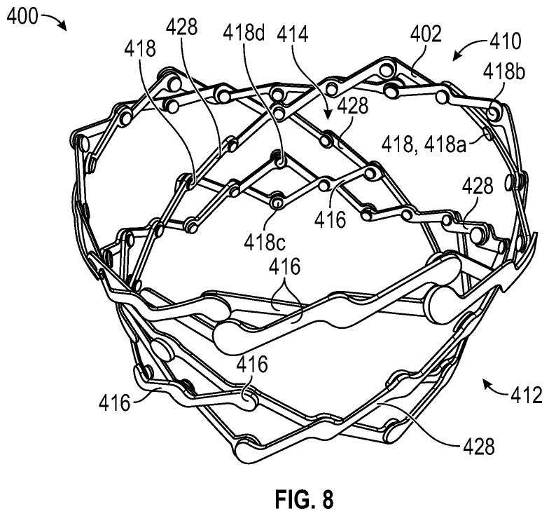

Referring now to FIG. 8, in another example, a docking device 400 can comprise a frame 402 having sub-frames 410, 412 connected by linking portion 414. Docking device 400 can be similar to docking device 200 described previously and can comprise one or more expansion and locking mechanisms configured to actuate (e.g., radially expand or compress) the first sub-frame 410 and the second sub-frame 412 independently of one another. Each sub-frame 410, 412 can comprise a plurality of struts 416 coupled together at junctions 418 and the sub-frames 410, 412 can be linked together via linking struts 428.

A first expansion and locking mechanism (e.g., similar to expansion and locking mechanism 208 described previously) can be coupled to the first sub-frame 410 and can radially expand and/or compress the first sub-frame 410 independently of the second sub-frame 412. The expansion and locking mechanism can have an inner member (e.g., similar to inner member 234) coupled to a first junction 418a of the first sub-frame 410 and an outer member (e.g., similar to outer member 232) coupled to a second junction 418b of the first sub-frame 410. To allow independent actuation of the first sub-frame 410 without corresponding actuation of the second sub-frame 412, the junctions 418a, 418b to which the expansion and locking mechanism is coupled do not comprise linking struts 428. For example, as shown in the illustrated example, neither junction 418a nor junction 418b comprises a linking strut 428. A second expansion and locking mechanism (e.g., similar to expansion and locking mechanism 208 described previously) can have an inner member coupled to a first junction 418c of the second sub-frame 412 and an outer member coupled to a second junction 418d of the second sub-frame 412. To allow independent actuation of the second sub-frame 412, the junctions 418c, 418b do not comprising linking struts 428.

Such a configuration allows the first sub-frame 410 to be expanded using the first expansion and locking mechanism while the second sub-frame 412 remains compressed, or vice versa. This advantageously allows additional flexibility during the implantation process. For example, a physician may expand and lock one sub-frame at a selected implantation site while retaining the other sub-frame in an unlocked state such that it can be further manipulated.

In some examples, a docking device such as docking devices 200 and/or 400 can include a plurality of expansion and locking mechanisms configured to allow either independent or simultaneous actuation of the first and second sub-frames. For example, a docking device can comprise an expansion and locking mechanism coupled to two junctions, each of which comprises at least one linking strut, and can further comprise one or more expansion mechanisms each coupled to two junctions at least one of which does not comprise a linking strut.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A docking device, comprising:
a radially expandable and compressible frame comprising:
   a first sub-frame comprising a first plurality of struts pivotably coupled to one another,
   a second sub-frame comprising a second plurality of struts pivotably coupled to one another, and
   one or more linking struts extending from a first end of the frame to a second end of the frame and coupling the first sub-frame and the second sub-frame to one another; and
an actuator comprising a first member coupled to the frame at a first location and a second member coupled to the frame at a second location axially spaced from the first location.

Example 2. The docking device of any example herein, particularly example 1, wherein the first member is coupled to the second sub-frame at a location adjacent an outflow end portion of the second sub-frame, and wherein the second member is coupled to the second sub-frame at a location adjacent an inflow end portion of the second sub-frame.

Example 3. The docking device of any example herein, particularly any one of examples 1-2, wherein the actuator is a first actuator and wherein the docking device further comprises a second actuator comprising a first member coupled to the first sub-frame at a location adjacent an inflow end portion of the first sub-frame and a second member coupled to the first sub-frame at a location adjacent an outflow end portion of the first sub-frame.

Example 4. The docking device of any example herein, particularly any one of examples 1-3, wherein the first and second sub-frames are radially expandable independently from one another.

Example 5. The docking device of any example herein, particularly examples 1-2, wherein the first member is coupled to a first junction comprising a first linking strut and the second member is coupled to a second junction comprising a second linking strut.

Example 6. The docking device of any example herein, particularly example 5, wherein movement of the first member relative to the second member in a first direction causes simultaneous radial expansion of the first and second sub-frames.

Example 7. The docking device of any example herein, particularly any one of examples 1-2, wherein the first member is coupled to a first junction on the first sub-frame comprising a first linking strut and wherein the second member is coupled to a second junction on the first sub-frame comprising a second linking strut.

Example 8. The docking device of any example herein, particularly any one of examples 1-7, wherein the first member extends at least partially into the second member.

Example 9. The docking device of any example herein, particularly any one of examples 1-8, wherein the first and second sub-frames are spaced axially apart from one another.

Example 10. The docking device of any example herein, particularly any one of examples 1-9, wherein the first and second pluralities of struts extend less than a full length of the frame.

Example 11. The docking device of any example herein, particularly any one of examples 1-10, wherein the docking device comprises three actuators disposed circumferentially around the frame.

Example 12. The docking device of any example herein, particularly any one of examples 1-11, wherein each sub-frame comprises a single row of cells extending circumferentially around the frame.

Example 13. The docking device of any example herein, particularly any one of examples 1-12, wherein a first subset of linking struts extends in a first direction and a second subset of linking struts extends in a second direction such that one or more struts of the first and second subsets overlap at one or more inflow apices of the first sub-frame.

Example 14. The docking device of any example herein, particularly any one of examples 1-13, further comprising a valvular structure disposed within and coupled to at least one of the first and second sub-frame.

Example 15. The docking device of any example herein, particularly any one of examples 1-13, further comprising a prosthetic valve disposed within the second sub-frame, the prosthetic valve comprising a radially expandable and compressible frame and a valvular structure disposed within and coupled to the frame.

Example 16. The docking device of any example herein, particularly any one of examples 1-15, further comprising a prosthetic valve disposed within the first sub-frame, the prosthetic valve comprising a radially expandable and compressible frame and a valvular structure disposed within and coupled to the frame.

Example 17. The docking device of any example herein, particularly any one of examples 1-16, further comprising a sealing member disposed on an outer surface of at least one of the first and second sub-frames, the sealing member configured to expand radially to secure the docking device at a selected implantation site.

Example 18. The docking device of any example herein, particularly any one of examples 1-17, wherein the actuator further comprises a locking member configured to lock the frame in an expanded configuration.

Example 19. A docking device, comprising:
a radially expandable and compressible frame including first and second sub-frames spaced axially apart from one another, each sub-frame comprising a plurality of struts pivotably coupled to one another at a plurality of junctions defining a single row of cells extending circumferentially around the frame, wherein the plurality of struts comprises one or more linking struts extending from an inflow end of the frame to an outflow end of the frame and coupling the first and second sub-frames to one another; and
one or more expansion and locking mechanisms each comprising an outer member coupled to the second sub-frame at a first junction, an inner member coupled to the second sub-frame at a second junction axially spaced from the first junction, and a locking member configured to retain the second sub-frame in an expanded configuration.

Example 20. The docking device of any example herein, particularly example 19, wherein the first and second junctions each comprise a linking strut.

Example 21. The docking device of any example herein, particularly example 20, wherein movement of the outer member relative to the inner member in a first direction causes simultaneous radial expansion of the first and second sub-frames.

Example 22. The docking device of any example herein, particularly any one of examples 19-21, further comprising a prosthetic valve disposed within at least one of the first sub-frame and the second sub-frame, the prosthetic valve comprising a radially expandable and compressible frame and a valvular structure disposed within and coupled to the frame.

Example 23. The docking device of any example herein, particularly any one of examples 19-22, further comprising a sealing member disposed on an outer surface of at least one of the first and second sub-frames, the sealing member configured to expand radially to secure the docking device at a selected implantation site.

Example 24. The docking device of any example herein, particularly any one of examples 19-23, wherein the docking device comprises three expansion and locking mechanisms disposed circumferentially around the frame.

Example 25. The docking device of any example herein, particularly any one of examples 19-24, wherein a first subset of linking struts extends in a first direction and a second subset of linking struts extends in a second direction such that one or more struts of the first and second subsets overlap at one or more outflow apices of the first sub-frame.

Example 26. A docking device, comprising:
a radially expandable and compressible frame including a controlling sub-frame and a controlled sub-frame coupled to the controlling sub-frame via one or more linking struts extending from an inflow end of the frame to an outflow end of the frame;
an actuator comprising a first member coupled to the controlling sub-frame at a first location and a second member coupled to the controlling sub-frame at a second location axially spaced from the first location;
wherein movement of the first member and the second member relative to one another in a first direction applies an expansion force to the controlling sub-frame to cause radial expansion of the controlling sub-frame; and
wherein the linking struts are configured to transfer the expansion force to the controlled sub-frame such that the controlling sub-frame and the controlled sub-frame expand simultaneously.

Example 27. The docking device any example herein, particularly example 26, wherein the first member is coupled to the controlling sub-frame at a location adjacent an outflow end portion of the controlling sub-frame, and wherein the second member is coupled to the controlling sub-frame at a location adjacent an inflow end portion of the controlling sub-frame.

Example 28. The docking device of any example herein, particularly any one of examples 26-27, wherein the first member is coupled to the controlling sub-frame at a first junction comprising a first linking strut and the second member is coupled to the controlling sub-frame at a second junction comprising a second linking strut.

Example 29. The docking device of any example herein, particularly any one of examples 26-28, wherein the first member extends at least partially into the second member.

Example 30. The docking device of any example herein, particularly any one of examples 26-29, wherein the controlled and controlling sub-frames are spaced axially apart from one another.

Example 31. The docking device of any example herein, particularly any one of examples 26-30, wherein the controlling and controlled sub-frames each comprise a plurality of struts extending less than a full length of the frame.

Example 32. The docking device of any example herein, particularly any one of examples 26-31, wherein the docking device comprises three actuators disposed circumferentially around the frame.

Example 33. The docking device of any example herein, particularly any one of examples 26-32, wherein each sub-frame comprises a single row of cells extending circumferentially around the frame.

Example 34. The docking device of any example herein, particularly any one of examples 26-33, wherein a first subset of linking struts extends in a first direction and a second subset of linking struts extends in a second direction such that one or more struts of the first and second subsets overlap at one or more outflow apices of the controlled sub-frame.

Example 35. The docking device of any example herein, particularly any one of examples 26-34, further comprising a prosthetic valve disposed within the controlling sub-frame, the prosthetic valve comprising a radially expandable and compressible frame and a valvular structure disposed within and coupled to the frame.

Example 36. The docking device of any example herein, particularly any one of examples 26-34, further comprising a sealing member disposed on an outer surface of at least one of the sub-frames, the sealing member configured to expand radially to secure the docking device at a selected implantation site.

Example 37. The docking device of any example herein, particularly any one of examples 26-35, wherein the actuator further comprises a locking member configured to lock the frame in an expanded configuration.

Example 38. A docking device, comprising:
a radially expandable and compressible frame comprising:
   a first sub-frame comprising a first plurality of struts pivotably coupled to one another,
   a second sub-frame comprising a second plurality of struts pivotably coupled to one another, and
   one or more linking struts extending from an inflow end of the frame to an outflow end of the frame and coupling the first sub-frame and the second sub-frame to one another;
   first and second expansion and locking mechanisms each comprising an outer member and an inner member extending at least partially into the outer member; and
wherein the first expansion and locking mechanism is coupled to the first sub-frame and the second expansion and locking mechanism is coupled to the second sub-frame such that the first and second sub-frames are radially expandable and compressible independently from one another.

Example 39. An assembly, comprising:
a docking device, comprising:
   a radially expandable and compressible frame including first and second sub-frames spaced axially apart from one another, each sub-frame comprising a plurality of struts pivotably coupled to one another at a plurality of junctions defining a single row of cells extending circumferentially around the frame, wherein the plurality of struts comprises one or more linking struts extending from an inflow end of the first sub-frame to an inflow end of the second sub-frame and coupling the first and second sub-frames to one another, and
   one or more expansion and locking mechanisms each comprising an outer member coupled to the second sub-frame at a first junction, an inner member coupled to the second sub-frame at a second junction axially spaced from the first junction, and a locking member configured to retain the second sub-frame in an expanded configuration; and a delivery apparatus, comprising:
   a handle,
   a first actuation member extending from the handle and coupled to the outer member, the first actuation member configured to apply a first expansion force to the first member, and
   a second actuation member extending from the handle and coupled to the inner member, the second actuation member configured to apply a second expansion force to the second member,
wherein application of the first and second expansion forces via the first and second actuation members causes radial expansion of the second sub-frame and the first sub-frame simultaneously.

Example 40. The assembly of any example herein, particularly example 39, wherein the outer member of the expansion and locking mechanism is coupled to a first junction comprising a first linking strut and the inner member is coupled to a second junction comprising a second linking strut.

Example 41. The assembly of any example herein, particularly any one of examples 39-40, wherein the outer member is coupled to the second sub-frame at a location adjacent an outflow end portion of the second sub-frame, and wherein the inner member is coupled to the second sub-frame at a location adjacent an inflow end portion of the second sub-frame.

Example 42. An assembly, comprising:
a docking device, comprising:
a radially expandable and compressible frame including first and second sub-frames spaced axially apart from one another, each sub-frame comprising a plurality of struts pivotably coupled to one another at a plurality of junctions defining a single row of cells extending circumferentially around the frame, wherein the plurality of struts comprises one or more linking struts extending from an inflow end of the first sub-frame to an inflow end of the second sub-frame and coupling the first and second sub-frames to one another, and
first and second expansion and locking mechanisms each comprising an outer member and an inner member extending at least partially into the outer member, wherein the first expansion and locking mechanism is coupled to the first sub-frame and the second expansion and locking mechanism is coupled to the second sub-frame;
a delivery apparatus, comprising:
   a handle,
   first and second actuation assemblies extending from the handle coupled to respective expansion and locking mechanism, each actuation assembly configured to apply an expansion force to a respective expansion and locking mechanism;
wherein application of a first expansion force to the first sub-frame via the first actuation assembly causes radial expansion of the first sub-frame independently of the second sub-frame; and
wherein application of a second expansion force to the second sub-frame via the second actuation assembly causes radial expansion of the second sub-frame independently of the first sub-frame.

Example 43. The assembly of any example herein, particularly example 42, wherein the outer member of the first expansion and locking mechanism is coupled to the first sub-frame at a location adjacent an inflow end portion of the first sub-frame, and wherein the inner member of the first expansion and locking mechanism is coupled to the first sub-frame at a location adjacent an outflow end portion of the first sub-frame.

Example 44. The assembly of any example herein, particularly any one of examples 42-43, wherein the outer member of the second expansion and locking mechanism is coupled to the second sub-frame at a location adjacent an outflow end portion of the second sub-frame, and wherein the inner member of the second expansion and locking mechanism is coupled to the second sub-frame at a location adjacent an inflow end portion of the second sub-frame.

Example 45. An assembly, comprising:
a docking device, comprising:
   a radially expandable and compressible frame including first and second sub-frames spaced axially apart from one another, each sub-frame comprising a plurality of struts pivotably coupled to one another at a plurality of junctions defining a single row of cells extending circumferentially around the frame, wherein the plurality of struts comprises one or more linking struts extending from an inflow end of the first sub-frame to an inflow end of the second sub-frame and coupling the first and second sub-frames to one another, and
   one or more expansion and locking mechanisms each comprising an outer member coupled to the second sub-frame at a first junction, an inner member coupled to the second sub-frame at a second junction axially spaced from the first junction, and a locking member configured to retain the second sub-frame in an expanded configuration; and a delivery apparatus, comprising:
   a handle,
   a first actuation member extending from the handle and coupled to the outer member, the first actuation member configured to apply a first expansion force to the first member, and
   a second actuation member extending from the handle and coupled to the inner member, the second actuation member configured to apply a second expansion force to the second member, wherein application of the first and second expansion forces via the first and second actuation members causes radial expansion of the second sub-frame and the first sub-frame simultaneously; and
a prosthetic valve disposed within the second sub-frame, the prosthetic valve comprising a radially expandable and compressible frame and a valvular structure disposed within and coupled to the frame.

Example 46. A method, comprising:
inserting a distal end portion of a delivery apparatus into a vasculature of a patient, the delivery apparatus releasably coupled to a docking device movable between a radially compressed and a radially expanded configuration, the docking device comprising a frame including first and second sub-frames spaced axially apart from one another and one or more actuators, each sub-frame comprising a plurality of struts pivotably coupled to one another at a plurality of junctions and including one or more linking struts extending from an inflow end of the first sub-frame to an inflow end of the second sub-frame and coupling the first and second sub-frames to one another;
advancing the docking device to a selected implantation site; and
actuating the actuators to radially expand the second sub-frame such that the expansion force is transferred to the first sub-frame via the linking struts allowing the first and second sub-frames to expand simultaneously.

Example 47. The method of any example herein, particularly example 46, wherein each actuator comprises an outer member coupled to the second sub-frame at a first junction comprising a first linking strut, and an inner member coupled to the second sub-frame at a second junction comprising a second linking strut and axially spaced from the first junction, and wherein actuating the actuators comprises moving the outer member and the inner member relative to one another.

Example 48. The method of any example herein, particularly any one of examples 46-47, wherein the selected implantation site is within a patient's heart such that the first sub-frame is disposed within the superior vena cava and the second sub-frame is disposed within the inferior vena cava.

Example 49. The method of any example herein, particularly any one of examples 46-47, further comprising:
inserting a distal end portion of a delivery apparatus into a vasculature of the patient, the delivery apparatus releasably coupled to a prosthetic valve movable between a radially compressed and a radially expanded configuration, the prosthetic valve comprising a frame and a valvular structure disposed within the frame;
advancing the prosthetic valve to a selected implantation site within the docking device; and
radially expanding the prosthetic valve.

Example 50. The method of any example herein, particularly example 49, wherein the selected implantation site is within the first sub-frame.

Example 51. The method of any example herein, particularly example 49, wherein the selected implantation site is within the second sub-frame.

Example 52. A docking device, comprising:
a radially expandable and compressible frame comprising:
   a first sub-frame comprising a first plurality of struts extending less than a full distance from a first end of the frame to a second end of the frame;
   a second sub-frame comprising a second plurality of struts extending less than a full distance from the first end of the frame to the second end of the frame, the second plurality of struts being spaced axially apart from the first plurality of struts; and
   one or more linking struts extending from the first end of the frame to the second end of the frame and coupling the first sub-frame and the second sub-frame to one another.

Example 53. The docking device of any example herein, particularly example 52, further comprising an actuator comprising a first member coupled to the frame at a first location and a second member coupled to the frame at a second location axially spaced from the first location.

Example 54. The docking device of any example herein, particularly example 53, wherein the first member is coupled to the second sub-frame at a location adjacent an outflow end portion of the second sub-frame, and wherein the second member is coupled to the second sub-frame at a location adjacent an inflow end portion of the second sub-frame.

Example 55. The docking device of any example herein, particularly any one of examples 52-54, wherein the first and second sub-frames are radially expandable independently from one another.

Example 56. The docking device of any example herein, particularly any one of examples 53-55, wherein the first member is coupled to a first junction comprising a first linking strut and the second member is coupled to a second junction comprising a second linking strut.

Example 57. The docking device of any example herein, particularly example 56, wherein movement of the first member relative to the second member in a first direction causes simultaneous radial expansion of the first and second sub-frames.

Example 58. The docking device of any example herein, particularly any one of examples 53-57, wherein the first member is coupled to a first junction on the first sub-frame comprising a first linking strut and wherein the second member is coupled to a second junction on the first sub-frame comprising a second linking strut.

Example 59. The docking device of any example herein, particularly any one of examples 52-58, wherein each sub-frame comprises a single row of cells extending circumferentially around the frame.

Example 60. The docking device of any example herein, particularly any one of examples 52-59, wherein a first subset of linking struts extends in a first direction and a second subset of linking struts extends in a second direction such that one or more struts of the first and second subsets overlap at one or more inflow apices of the first sub-frame.

Example 61. The docking device of any example herein, particularly any one of examples 52-60, further comprising a valvular structure disposed within and coupled to at least one of the first and second sub-frame.

Example 62. The docking device of any example herein, particularly any one of examples 52-62, further comprising a prosthetic valve disposed within the second sub-frame, the prosthetic valve comprising a radially expandable and compressible frame and a valvular structure disposed within and coupled to the frame.

Example 63. The docking device of any example herein, particularly any one of examples 52-62, further comprising a prosthetic valve disposed within the first sub-frame, the prosthetic valve comprising a radially expandable and compressible frame and a valvular structure disposed within and coupled to the frame.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A docking device, comprising:
a radially expandable and compressible frame comprising:
a first sub-frame comprising a first plurality of struts pivotably coupled to one another,
a second sub-frame comprising a second plurality of struts pivotably coupled to one another, and
one or more linking struts extending from a first end of the frame to a second end of the frame and coupling the first sub-frame and the second sub-frame to one another; and
an actuator comprising a first member coupled to the frame at a first location and a second member coupled to the frame at a second location axially spaced from the first location.

2. The docking device of claim 1, wherein the first member is coupled to the second sub-frame at a location adjacent an outflow end portion of the second sub-frame, and wherein the second member is coupled to the second sub-frame at a location adjacent an inflow end portion of the second sub-frame.

3. The docking device of claim 1, wherein the actuator is a first actuator and wherein the docking device further comprises a second actuator comprising a first member coupled to the first sub-frame at a location adjacent an inflow end portion of the first sub-frame and a second member coupled to the first sub-frame at a location adjacent an outflow end portion of the first sub-frame.

4. The docking device of claim 1, wherein the first and second sub-frames are radially expandable independently from one another.

5. The docking device of claim 1, wherein the first member is coupled to a first junction comprising a first linking strut and the second member is coupled to a second junction comprising a second linking strut.

6. The docking device of claim 5, wherein movement of the first member relative to the second member in a first direction causes simultaneous radial expansion of the first and second sub-frames.

7. The docking device of claim 1, wherein the first member is coupled to a first junction on the first sub-frame comprising a first linking strut and wherein the second member is coupled to a second junction on the first sub-frame comprising a second linking strut.

8. The docking device of claim 1, wherein the first member extends at least partially into the second member.

9. The docking device of claim 1, wherein the first and second sub-frames are spaced axially apart from one another.

10. The docking device of claim 1, wherein the first and second pluralities of struts extend less than a full length of the frame.

11. The docking device of claim 1, wherein the docking device comprises three actuators disposed circumferentially around the frame.

12. The docking device of claim 1, wherein each sub-frame comprises a single row of cells extending circumferentially around the frame.

13. The docking device of claim 1, wherein a first subset of linking struts extends in a first direction and a second subset of linking struts extends in a second direction such that one or more struts of the first and second subsets overlap at one or more inflow apices of the first sub-frame.

14. The docking device of claim 1, further comprising a valvular structure disposed within and coupled to at least one of the first and second sub-frame.

15. The docking device of claim 1, further comprising a prosthetic valve disposed within the second sub-frame, the prosthetic valve comprising a radially expandable and compressible frame and a valvular structure disposed within and coupled to the frame.

16. The docking device of claim 1, further comprising a prosthetic valve disposed within the first sub-frame, the prosthetic valve comprising a radially expandable and compressible frame and a valvular structure disposed within and coupled to the frame.

17. The docking device of claim 1, further comprising a sealing member disposed on an outer surface of at least one of the first and second sub-frames, the sealing member configured to expand radially to secure the docking device at a selected implantation site.

18. The docking device of claim 1, wherein the actuator further comprises a locking member configured to lock the frame in an expanded configuration.

19. A docking device, comprising:
a radially expandable and compressible frame including first and second sub-frames spaced axially apart from one another, each sub-frame comprising a plurality of struts pivotably coupled to one another at a plurality of junctions defining a single row of cells extending circumferentially around the frame, wherein the plurality of struts comprises one or more linking struts extending from a first end of the frame to a second end of the frame and coupling the first and second sub-frames to one another; and
one or more expansion and locking mechanisms each comprising an outer member coupled to the second sub-frame at a first junction, an inner member coupled to the second sub-frame at a second junction axially spaced from the first junction, and a locking member configured to retain the second sub-frame in an expanded configuration.

20. The docking device of claim 19, wherein the first and second junctions each comprise a linking strut.

21. The docking device of claim 20, wherein movement of the outer member relative to the inner member in a first direction causes simultaneous radial expansion of the first and second sub-frames.

22. The docking device of claim 19, further comprising a prosthetic valve disposed within at least one of the first sub-frame and the second sub-frame, the prosthetic valve comprising a radially expandable and compressible frame and a valvular structure disposed within and coupled to the frame.

23. The docking device of claim 19, further comprising a sealing member disposed on an outer surface of at least one of the first and second sub-frames, the sealing member configured to expand radially to secure the docking device at a selected implantation site.

24. The docking device of claim 19, wherein the docking device comprises three expansion and locking mechanisms disposed circumferentially around the frame.

25. The docking device of claim 19, wherein a first subset of linking struts extends in a first direction and a second subset of linking struts extends in a second direction such that one or more struts of the first and second subsets overlap at one or more outflow apices of the first sub-frame.
